(19) United States
(12) Patent Application Publication
(10) Pub. No.: US 2008/0091181 A1
(43) Pub. Date: Apr. 17, 2008

(54) System and method for ablation

(76) Inventor: Shlomo Gabbay, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/546,154

(22) Filed: Oct. 11, 2006

(51) Int. Cl. A61B 18/04

(52) U.S. Cl. 606/27

(57) ABSTRACT

A system for providing ablation comprising a fluid source that contains a volume of biocompatible ablation fluid. The system also comprises a heater that heats the ablation fluid and provides heated ablation fluid. The system further comprises an ablation applicator, spaced apart from the heater, and having a perimeter edge configured to engage tissue surround an area of tissue, the ablation applicator applying ablation fluid to the area of tissue. The system still further comprises a suction source for removing the ablation fluid from the area of tissue.

### TECHNICAL FIELD

The present invention relates to a system and method of ablation in surgical procedures.

### BACKGROUND

Atrial fibrillation results from disorganized electrical activity in the heart muscle, or myocardium. A surgical maze procedure has been developed for treating atrial fibrillation and the procedure involves the creation of a series of surgical incisions through the atrial myocardium in a preselected pattern so as to create conductive corridors of viable tissue bounded by scar tissue.

As an alternative to the surgical incisions used in the maze procedure, transmural ablation of the heart wall has been proposed. Such ablation may be performed either from within the chambers of the heart (endocardial ablation) using endovascular devices (e.g., catheters) introduced through arteries or veins, or from outside the heart (epicardial ablation) using devices introduced into the chest. Various ablation technologies have been proposed, including cryogenic, radiofrequency (RF), laser and microwave. The ablation devices are used to create transmural lesions, that is, lesions extending through a sufficient thickness of the myocardium to block electrical conduction, which form the boundaries of the conductive corridors in the atrial myocardium. Perhaps most advantageous about the use of transmural ablation rather than surgical incisions is the ability to perform the procedure on the beating heart without the use of cardiopulmonary bypass.

### SUMMARY

One aspect of the present invention relates to a system for providing ablation comprising a fluid source that contains a volume of biocompatible ablation fluid. The system also comprises a heater that heats the ablation fluid and provides heated ablation fluid. The system further comprises an ablation applicator, spaced apart from the heater, and having a perimeter edge configured to engage tissue and surround an area of tissue, the ablation applicator applies ablation fluid to the area of tissue. The system still further comprises a suction source for removing the ablation fluid from the area of tissue.

Another aspect of the present invention relates to a method for performing ablation comprising heating, by a heat source, ablation fluid to a predetermined temperature. The method also comprises providing the heated ablation fluid to an ablation applicator that is spaced apart from the heat source by a conduit. The method further comprises ablating a tissue area of a patient located within a volume define by the tissue and the ablation applicator. The method still further comprises removing, by a suction source, heated ablation fluid from the volume.

Still another aspect of the present invention relates to a system for providing ablation comprising means for holding/containing a volume of an ablation fluid. The system also comprises means for heating a portion of the ablation fluid. The system further comprises means for applying the heated ablation fluid that is spaced apart from the means for heating to a surface area define by an area of tissue surrounded by the means for applying. The system still further comprises means for applying negative pressure to remove the heated ablation fluid from the surface area.

### BRIEF DESCRIPTION OF THE DRAWINGS

FIG. 1 illustrates an example of an ablation system in accordance with an aspect of the invention.

FIG. 2 illustrates an example of an ablation applicator in accordance with an aspect of the invention.

FIG. 3 illustrates another example of an ablation applicator in accordance with an aspect of the invention.

FIG. 4 illustrates another example of an ablation applicator in accordance with an aspect of the invention.

FIG. 5 illustrates an example of an ablation procedure performed on a heart in accordance with an aspect of the invention.

### DETAILED DESCRIPTION

The present invention relates to a system and method for ablating tissue. The heating of the tissue kills the tissue, while leaving the surrounding tissue unharmed.

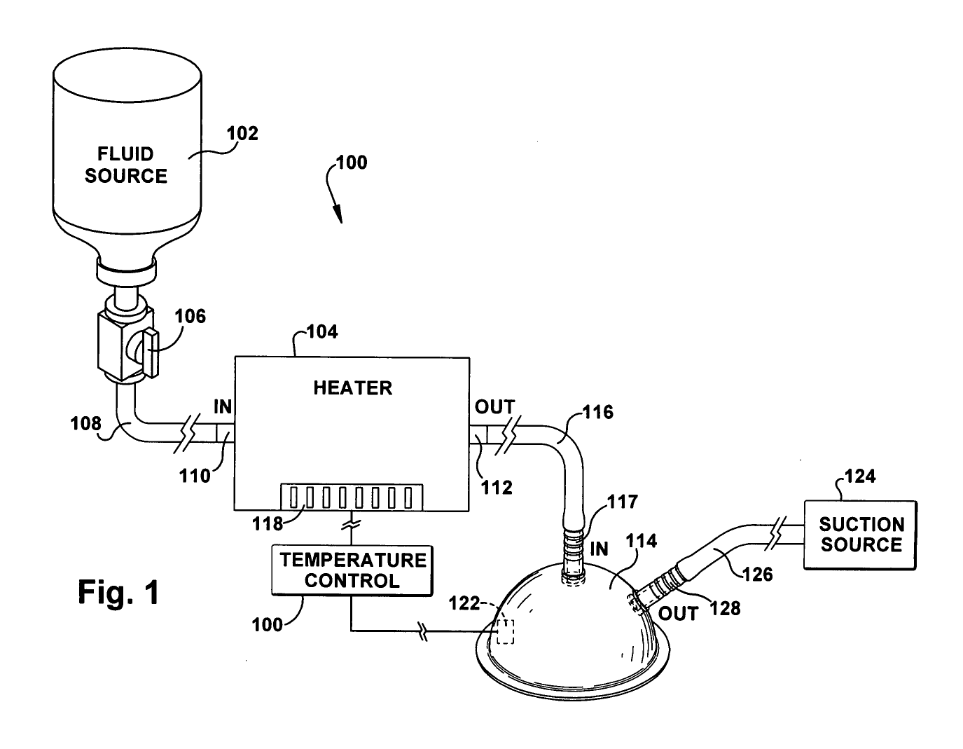

FIG. 1 illustrates an example of an ablation system 100 in accordance with an aspect of the invention. The ablation system 100 includes an ablation fluid source 102 that provides ablation fluid for use in ablation tissue. The fluid from the source 102 is provided to a heater 104. The ablation fluid can be, for example, a substantially sterile saline solution or other biocompatible fluid. The ablation fluid source 102 can be implemented as a passive container that holds the fluid, such as for example, as a drip bag, which can employ gravity to supply the fluid or fluid can be retrieved by a pump or other device. Alternatively, the source 102 can include a pump that supplies the fluid to the heater 104. Thusly, the ablation fluid source 102 can provide means for holding/containing a volume of the ablation fluid. The ablation fluid source 102 can include, for example, an adjustable valve 106, such as a ball valve, gate valve or globe valve. The valve 106 can include, for example, an OFF setting that prevents the flow of ablation fluid, and an ON setting that allows the flow of ablation fluid. The valve 106 can, for example, the rate of flow of ablation fluid provided by the ablation fluid source 102 by a setting between the ON and OFF settings. Alternative flow can be controlled by electrical or mechanical control of an associated pump.

The ablation fluid source 102 can be connected to the heater 104, for example, by one or more lengths of a flexible tube 108 or other conduit. The flexible tube 108 could be implemented, for example, as a insulated or non-insulated silicone tube. The flexible tube 108 could be implemented as a separate component from the ablation fluid source 102, having first and second ends. In such an example, the first end of the flexible tube 108 could be removeably connected to an outlet or part of the source 102 such as or to the adjustable valve 106 of the ablation fluid source 102. The second end of the flexible tube 108 can be removeably connected to the input port 110 the heater 104. Alternatively, the flexible tube 108 could be integrally formed on the ablation fluid source 102, wherein an end of the flexible tube 108 is connected to an input port 110 of the heater 104. It is to be understood that integral formation includes monolithic formation processes, such as injection molding. The ablation fluid can flow within a lumen of the flexible tube 108 from the ablation fluid source 102 to the input port 110 of the heater 104.

The heater 104 can receive the ablation fluid at the input port 110, heat the ablation fluid and provide heated ablation fluid at an output port 112. The heated ablation fluid can be supplied to an ablation applicator 114 for use in ablating selected tissue. The ablation applicator 114 can be connected to the heater by a connector tube 116. The heater 104 can include one or more heating elements 118 that can be controlled, for example, by a temperature controller 120.

As one example, the heater 104 can include a chamber for heating the liquid from the fluid source 102. As one example, the chamber can store heating liquid, such as water, or other fluid with a relatively high specific heat (e.g., a specific heat of at least about 4 Joules per gram per degree Celsius). The heater 104 can also include an immersion tube, for example, which can be implemented as elongated tube that is immersed in the heating liquid. The immersion tube can connect the input port 110 to the output port 112 of the heater 104, such that ablation fluid can flow within a lumen of the immersion tube from the input port 110 to the output port 112 of the heater 104. The immersion tube can, for example, be formed out of a conductive material, such as copper or aluminum, which is heated by the one or more heating elements 118. The immersion tube can, for example, be shaped as a coil such that the ablation fluid flowing through in the immersion tube can be heated by the heating fluid. Thusly, the heating fluid and the immersion tube can act as a medium of heat transfer between the heating element 118 and the ablation fluid flowing in the lumen of the immersion tube. Upon exiting the heater 104 through the output port 112, the ablation fluid can have approximately the same temperature as the heating fluid. The heater 104 thus can provide means for heating a portion of the ablation fluid.

As stated above, the heater 104 and the ablation applicator 114 can be connected by the connector tube 116. The connector tube 116 can be implemented, for example, as an elongated flexible tube or other insulated conduit. The connector tube 116 can include, for example, a first end a spaced apart from a second end. In such an example, the first end of the connector tube 116 could be removeably connected to the output port 112 of the heater 104 and the second end of the connector tube 116 could be removeably connected to an input port 117 of the ablation applicator 114. Alternatively, the connector tube 116 could be integrally formed with the ablation applicator 114. The connector tube 116 can be formed, for example, out of a compliant material, such as rubber, silicone or plastic to name a few. It is to be understood that, as used herein, the term rubber includes any of a variety of synthetic and/or natural elastic materials whose properties resemble natural rubber. It is to be further understood that the term plastic includes any of a variety of polymers that are compliant at room temperature The connector tube 116 can cause fluid communication between the heater 104 and the ablation applicator 114, such that heated ablation fluid can flow in a lumen of the connector tube 116 from the heater 104 to the ablation applicator 114.

The ablation applicator 114 receives the heated ablation fluid and can directly apply the heated ablation fluid to living tissue. The area of tissue which comes into direct contact with the heated ablation fluid is referred to herein as an ablation site. The ablation site can be located either by a process called mapping, in which the ablation applicator is moved from spot to spot until the appropriate area is found. Alternatively, the applicator 114 can be positioned at the ablation site under direct vision (in the case of an open chest procedure). As one example, the applicator can be robotically positioned at a desired implantation site as part of a partially or fully automated procedure. The ablation applicator 114 can be formed, for example, out of a non-conductive material, such as rubber or silicone or plastic or a combination of materials. An interior of the ablation applicator 114 can hold a volume of the heated ablation fluid. For instance, an open end of the applicator 114 can have a perimeter dimensioned and configured to contact the heart so that the ablation site resides in an area defined by the open end of the applicator. Heated ablation fluid flows into the volume defined by the ablation site and the interior surface of the application 114. When the living tissue comes into direct contact with the heated ablation fluid, the tissue will heat. For instance, the ablation fluid can be heated to approximately 65 degrees Celsius, such that the cells contacted by the heated ablation fluid will be destroyed (or killed) upon contact. A layer of tissue below the surfaces can also be destroyed according to the temperature of the ablation fluid and the duration such fluid is applied to the tissue.

The system 100 can also include a temperature control system 120 that provides means for controlling the temperature of the heated ablation fluid at the ablation site. The temperature control system can include, for example, a temperature sensor 122. The temperature sensor 122 can be implemented, for example, as a thermistor, an electronic thermometer or other temperature sensor. The temperature sensor 122 can sense the temperature of the heated ablation fluid within the ablation applicator 114, or alternatively, the temperature sensor 122 can sense the temperature of tissue. The temperature sensor 122 can be implemented as part of the ablation applicator 114. Alternatively, the temperature sensor 122 can sense the temperature of tissue. As one example, the temperature sensor 122 can be integrally formed into a housing of the ablation applicator 114. The temperature sensor 122 can provide a signal to the temperature controller 120 that characterizes the temperature sensed by the temperature sensor 122. The temperature controller 120 can be implemented, for example, as a microcontroller, a thermocouple, or other software and/or hardware.

The temperature controller 120 can be programmable, such that a user of the ablation system 100 can set a desired temperature for the heated ablation fluid. The temperature controller 120 receives the signal from the temperature sensor 122 and adjusts the intensity of the heating element 118 of the heater 104 based on the received signal and the desired temperature. Accordingly, the temperature controller 120 can control the temperature of the heated ablation fluid leaving the heater 104. The temperature controller 120 can ensure that the temperature of the heated ablation fluid stays at or near the desired temperature when the ablation fluid is at the ablation site. In this way, the temperature control system can provide closed loop temperature control. The temperature can be fixed or it can be programmable.

A suction source 124 can be implemented to cause the flow of ablation fluid from the source to the ablation application 114. The suction source 124 and the ablation applicator 114 can be connected by a suction tube 126. The suction tube 126 can be implemented, for example, as an elongated flexible tube or other conduit. The suction tube 126 could be formed, for example, out of rubber, silicone or plastic. The suction tube 126 could be formed separate from the suction source 124 and the ablation applicator 114. In such an example, the suction tube 126 can include a first and second end, wherein the first end is removeably connected to the suction source 124 and the second end is removeably connected to an output port 128 of the ablation applicator 114. Alternatively, the suction tube 126 could be integrally formed on the ablation applicator 114, and an end of the suction tube 126 could be removeably connected to the suction source 124. In another alternative, the suction tube 126 could be integrally formed with the suction source 124, and an end of the suction tube 126 could be removeably connected to the output port 128 of the ablation applicator 114.

The suction source 124, via the suction tube 126 can apply a negative pressure of about −14 to about −103 kilopascals (approximately −2 to −15 pounds per square inch) at the output port 128 of the ablation applicator 114. Application of the negative pressure can create a partial vacuum to pull the heated ablation fluid from the ablation fluid source 102, through the heater 104, to within the ablation applicator 114, and from the ablation applicator 114 to a waste reservoir (not shown). It is to be understood that the waste reservoir could be implemented as a container within the suction source 124, or as separate container spaced apart from the suction source 124. Appropriate controls can be provided to selectively activate and deactivate the solution source 124 to apply heated ablation fluid to the desired site(s). The suction source 124 can thus provide means for applying negative pressure to cause flow of the ablation fluid into the applicator 114 and to remove the heated ablation fluid from the ablation site.

By way of further example, the ablation applicator 114 can be placed at a specific ablation site during surgery. The ablation applicator 114 can be placed at the ablation site, for example, robotically or endoscopically. Typically, while the ablation applicator 114 is being positioned at the ablation site the valve 106 of the ablation fluid source 102 can be set to an OFF setting or other controls can configure the system 100, such that no ablation fluid is flowing through the ablation system 100. When the ablation applicator 114 is positioned at a desired ablation site, the suction source 124 can be activated to establish a substantially airtight seal around an edge of the ablation applicator 114 and the area of tissue engaged with the ablation applicator 114.

When the substantially airtight seal is established, the adjustable valve 106 on the ablation fluid source 102 or other controls can be set to an ON setting, such that ablation fluid can flow through the ablation system 100. The negative pressure applied by the suction source 124 causes ablation fluid to flow from the ablation fluid source 102 to the heater 104 via the lumen of the flexible tube 108. The ablation fluid can then flow from the heater's 104 input port 110 to the heater's 104 output port 112, thereby heating the fluid to a desired temperature. The heated ablation fluid can flow from the output port 112 of the heater 104 to the input port 117 of the ablation applicator 114 via the lumen of the connector tube 116.

When the ablation applicator 114 receives heated ablation fluid at the input port 117 and the suction source 124 provides the negative pressure at the output port 128 of the ablation applicator, the ablation applicator 114 maintains the relatively airtight seal around the area of tissue covered by the ablation applicator 114. Such a seal can ensure that only a desired area (the ablation site) of tissue comes into contact with the heated ablation fluid, such that tissue that is not intended to be ablated is not harmed. Suction can be set to maintain a substantially constant volume of fluid within the applicator during ablation. For instance, the heated ablation fluid can substantially fill the volume defined by the interior of the ablation applicator 114 and the surface at the ablation site. In this way, the ablation applicator 114 can provide means for applying heated ablation fluid to the ablation site. The suction source 124 can remove the heated ablation fluid from the ablation applicator 114 via the lumen of the suction tube 126, thereby creating a relatively constant flow of heated ablation fluid in the ablation applicator 114.

The ablation system 100 of the present invention allows a surgeon to accurately control the temperature of an ablation fluid (e.g., a saline solution), which can be directly applied to a desired ablation site. The heated ablation fluid can be directly applied by the ablation applicator 114. For example, the ablation fluid is heated to a range of approximately 60-70 degrees Celsius, which is sufficient to kill cells in the tissue that the heated ablation fluid comes into direct contact with for a sufficient amount of time. Typically, this process can continue until the surgeon has ablated a desired amount of tissue.

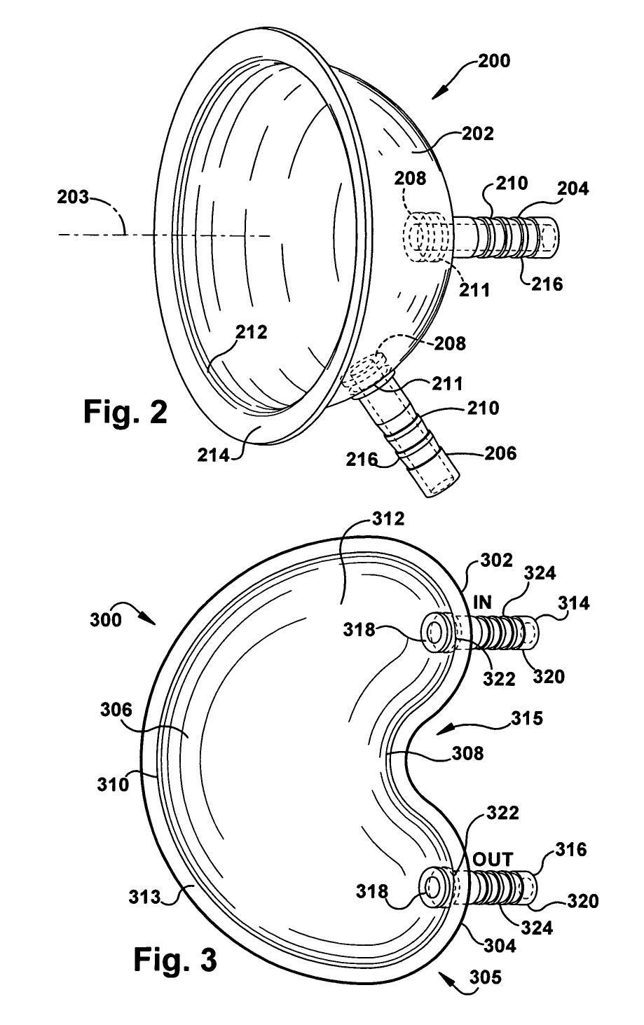

It is to be appreciated that the ablation applicator 114 could be formed in a variety or different size and shapes, as will be described herein. The size and shapes could depend, for example, on the size and shape of the tissue of anatomical structures that are to be ablated. In FIG. 1, the ablation applicator 114 illustrated includes a substantially circular shaped open contact edge, such as will be described in with respect to FIG. 2.

FIG. 2 illustrates an example of an ablation applicator 200 that can be used in the ablation system 100. The ablation applicator 200 can have substantially a hollow hemispherical housing wall 202 that forms a dome about an axis 203. The ablation applicator 200 can be formed of a non-conductive and flexible material, such as silicone, plastic or rubber or a combination of materials. The ablation applicator 200, could be formed, for example, by a monolithic injection molding process. The housing wall 202 of the ablation applicator 200 can be relatively thin (e.g., about 0.5-1.0 mm). The housing wall 202 of the ablation applicator 200 can be translucent, such that an observer of an ablation procedure can view an interior portion of the housing wall 202. This can allow proper placement of the application as well as provide a view of the flow of fluid.

An input port 204 and an output 206 can, for example, receive and provide heated ablation fluid, respectively, by allowing flow of the heated ablation fluid through a lumen of a respective input port 204 or output port 206. It is to be understood that the input port 204 and the output port 206 can be interchangeable. The input port 204 and the output port 206 can be formed of a relatively rigid material, such as thermoplastic polyethylene terephthalate (PET) or silicon. Alternatively, the input port 204 and the output port 206 can be formed of the same or similar material as the ablation applicator 200. The input port 204 and the output port 206 can include, for example, a first end 208 and a second end 210. The first end 208 of the input port 204 and the output port 206 can be internal to the ablation applicator 200. The second end 210 of the input port 204 and the output port 206 can, for example, be external to the ablation applicator 200. The input port 204 could, for example, be implemented as a first tube that extends from a proximal end of the housing wall 202 and axially along or parallel to the axis 203. The output port 206 can be implemented, for example, as a second tube that extends outwardly from the housing wall 202 and normal to the axis 203, and is spaced apart from the first tube.

The input port 204 and the output port 206 can be integrally formed with the ablation applicator 200. Alternatively, the input port 204 and the output port 206 can be attached to ablation applicator 200 by press fitting the input port 204 and the output port 206 through an aperture of the ablation applicator 200 up to a shoulder piece 211 of the input port 204 and the output port 206. The input port 204 and output port 206 can include, for example, a plurality of circumferentially extending ribs 216 or projections to provide increased friction fitting with attached tubes. Alternatively, the input port 204 and the output can be smooth.

The dome shaped portion of the housing wall 202 can, for example, define a volume. The volume can, for example, hold heated ablation fluid that is flowing from the input port 204 to the output port 206. A distal end of the ablation applicator 200 can, for example, form an open contact edge 212. The contact edge 212, can be for example, the portion of the ablation applicator 200 that engages tissue that surrounds an ablation site. The contact edge can lie substantially in a plane or have other contoured configurations. The contact edge 212 could be formed, for example, as a substantially circumferential opening. The contact edge 212 can include a ring portion 214 that can extend radially inwardly or outwardly from the axis 203. The ring portion 214 can, for example, increase the efficiency of the airtight seal mentioned above, by increasing the surface area of the contact edge 212.

FIG. 3 illustrates another example of a kidney-shaped ablation applicator 300 in accordance with an aspect of the invention. The ablation applicator 300 can, for example, be formed by a monolithic injection molding process. Additionally, the ablation applicator 300 can be formed out of a non-conductive and flexible material, such as silicone, plastic or rubber. The ablation applicator 300 can include a first end 302 and a second end 304 residing on a plane 305. The ablation applicator 300 can also include a middle portion 306 extending between the first end 302 and the second end 304. The middle portion 306 can include an inner surface 308 and an outer surface 310 that are spaced apart. The inner surface 308 and the outer surface 310 can, for example, extend from the first end 302 to the second end 304. The middle portion 306 can also include an arched portion 312 extending between the inner and outer surfaces 308 and 310. The arched portion 312 can, for example, have a substantially semicircular cross section. The arched portion 312 can be relatively thin (e.g., about 0.5 mm to about 1.0 mm). The arched portion 312 defines a housing wall of the ablation applicator 300. Additionally, the arched portion 312 can be substantially transparent, such as to allow an observer of an ablation procedure to view the interior of the arched portion 312. The ablation applicator 300 can be applied to ablation sites with an asymmetric surface, such as a surface of a heart.

The inner portion 308 and the outer portion 310 of the ablation applicator 300 can, for example, define a kidney-shaped contact edge 313. That is, a portion of the contact edge 313 between the spaced apart ends 302 and 304 can be recessed concavely, as indicated at 315. The contact edge 313 can be, for example, the portion of the ablation applicator 300 that engages tissue during an ablation procedure. The contact edge 313 can include, for example, a periphery portion extending inwardly or outwardly along the contact edge 313. The periphery portion can increase the efficiency of the airtight seal mentioned above, by increasing the surface area of the portion of the ablation applicator 300 that engages a patient's tissue. The periphery portion can also be more pliant then a proximal portion of the application 300 to facilitate its engagement at an ablation site.

The arched portion 312 of the ablation applicator 300 can define a volume for holding heated ablation fluid that flows from an input port 314 to an output port 316. The volume can, for example, define the ablation site. The ablation site can thusly include an area of tissue located within the contact edge 313 and the housing wall 312 of the ablation applicator 300.

The input port 314 and the output port 316 can, for example, receive and provide heated ablation fluid, respectively, by allowing flow of the heated ablation fluid through a lumen of a respective input port 314 or output port 316, similar to as described above with respect to FIG. 2. It is to be understood that the input port 314 and the output port 316 can be interchangeable. The input port 314 and the output port 316 can more rigid than the arched housing wall 312. The input port 314 and the output port 316 can be formed of the same or similar material as the ablation applicator 300. The input port 314 and the output port 316 can include, for example, a first end 318 and a second end 320. The first end 318 of the input port 314 and the output port 316, can for example, be internal to the ablation applicator 300. The second end 320 of the input port 314 and the output port 316 can, for example, be extend outwardly from the housing wall 312 of the ablation applicator 300 and can extend normally to the plane 305. The input port 314 and the output port 316 can be formed with the ablation applicator 300 similar to as mentioned with request to FIG. 2. The input port 314 and output port 316 can include, for example, a plurality of circumferentially extending ribs 324 or projections to provide increased friction fitting with attached tubes.

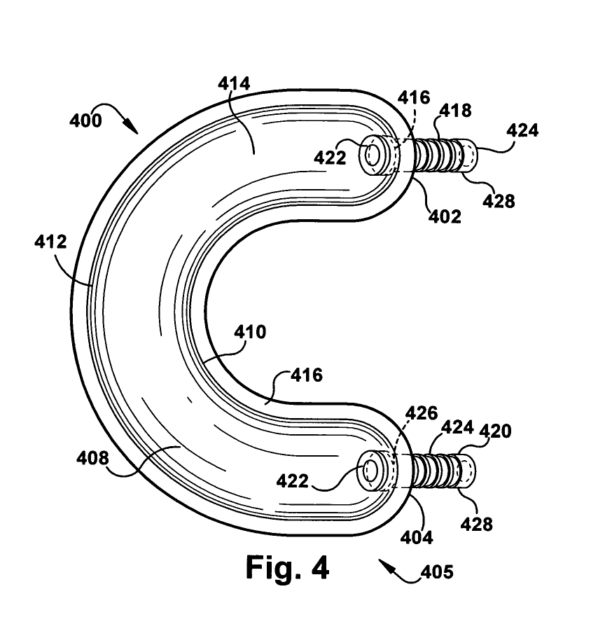

FIG. 4 illustrates another example of a substantially C-shaped ablation applicator 400 in accordance with an aspect of the invention. The ablation applicator 400 can, for example, be formed by a monolithic injection molding process. Additionally, the ablation applicator 400 can be formed out of a non-conductive and flexible material, such as silicone, plastic or rubber. The ablation applicator 400 can include, for example, a first end 402 and a second end 404 residing on a plane 406. The ablation applicator 400 can also include a middle body portion 408 extending arcuately between the first end 402 and the second end 404. The middle portion 408 can include, for example, follow a substantially hollow C-shape having an inner C-shaped surface 410 and outer C-shaped surface 412, wherein the inner surface 410 and outer surface 412 are spaced apart. The inner surface 410 and outer surface 412 can, for example, extend in a substantially fixed spaced apart relationship from the first end 402 to the second end 404. The middle portion 408 can also include an arched housing wall portion 414 extending arcuately between the inner and outer surfaces 410 and 412. The arched portion 414 can, for example, have a semicircular cross section. The arched portion 414 can be relatively thin (e.g., having a thickness of about 0.5 to about 1.0 millimeters). Additionally, the arched portion 414 can be translucent, such as to allow an observer of an ablation procedure to view the interior of the arched portion 414.

The inner surface 410 and the outer surface 412 of the ablation applicator 400 can define a contact edge 416 that engages tissue during an ablation procedure. The contact edge 416 can, for example, define a substantially hollow U- or C-shape such as shown in FIG. 4. The contact edge 416 can include, for example, a periphery portion extending inwardly or outwardly along the contact edge 416. The periphery portion can increase the efficiency of the airtight seal mentioned above, by increasing the surface area of the portion of the ablation applicator 400 that comes into contact with a patient's tissue.

The arched side wall portion 414 of the ablation applicator 400 can engage the ablation site to define a volume for holding heated ablation fluid that flows from an input port 418 to an output port 420.

The input port 418 and the output port 420 can, for example, receive and provide heated ablation fluid, respectively, by allowing flow of the heated ablation fluid through a lumen of a respective input port 418 or output port 420. It is to be understood that the input port 418 and the output port 420 can be interchangeable (i.e., fluid can flow in either direction). The input port 418 and the output port 420 can include, for example, a first end 422 and a second end 424. The first end 422 of the input port 418 and the output port 420, can for example, be internal to the ablation applicator 400. The second end 424 of the input port 418 and the output port 420 can extend outwardly from the respective ends 402 and 404 the ablation applicator 400. The input port 418 and output port 420 can extend outwardly from the first and second ends 402 and 404, respectively, of the ablation applicator 400 (e.g., extending normal to the plane 406). The input port 418 and output port 420 can include, for example, a plurality of circumferentially extending ribs 428 or projections to provide increased friction fitting with attached tubes.

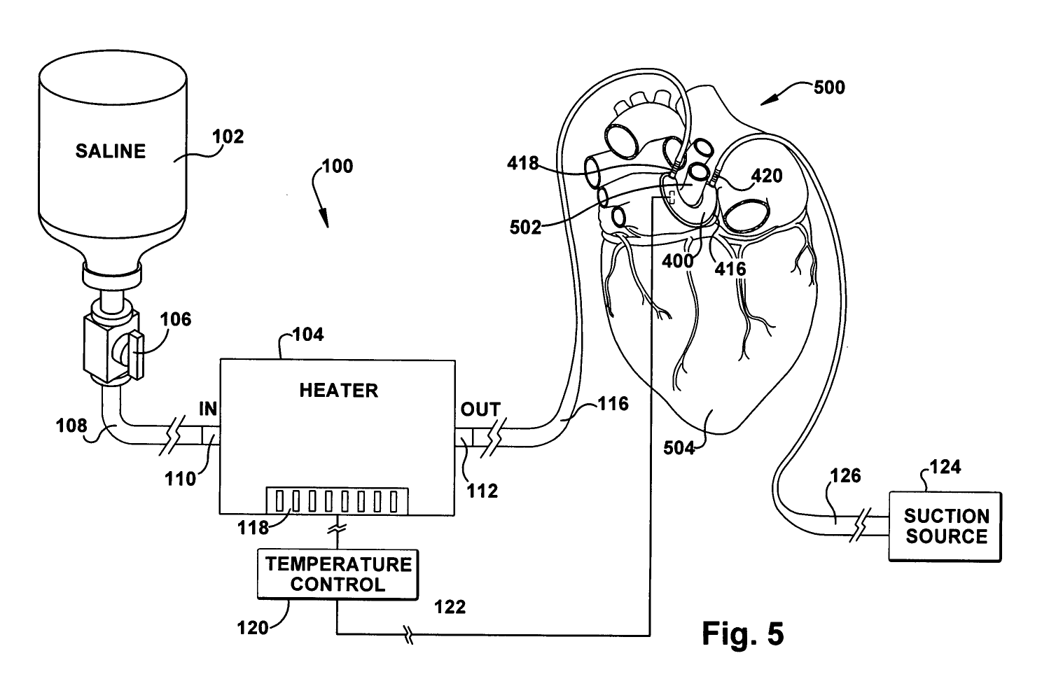

FIG. 5 illustrates an example of an ablation procedure 500 implementing the ablation system 100 illustrated and described herein with respect to FIG. 1. In this example, the ablation applicator 400 illustrated and described herein with respect to FIG. 4 is being applied about a pulmonic vein 502 of heart 504 in accordance with an aspect of the invention. For purposes of simplification of explanation, the same reference numbers will be used to identify parts previously introduced with respect to FIGS. 1 and 4. It to be understood that any of the ablation applicators 200, 300 and 400 illustrated and described herein could be used in a similar manner.

In the ablation procedure, the ablation applicator 400 is applied adjacent a lower portion of a right inferior pulmonic vein 502. In the present example, the ablation applicator 400 circumscribes at least a portion of the right inferior pulmonic vein 502. It is to be appreciated that the ablation applicator 400 could be applied to other surfaces of the heart, and the present procedure 500 disclosed is but one example implementation. The ablation applicator 400 could be applied to the right inferior pulmonic vein 506 by, for example, a robotic or endoscopic technique. Alternatively, the applicator 400 can be applied manually.

Initially, when the ablation applicator 400 is applied, the adjustable valve 106 of the ablation fluid source 102 can be set to the OFF position. Additionally, once ablation applicator 400 is in place, the suction source 124 can be activated. Activation of the suction source 124 creates an substantially airtight seal between the ablation applicator 400 and the ablation site (e.g., the heart tissue) around the right inferior pulmonic vein 506.

After a relatively short amount of time (e.g., 2-3 seconds), the adjustable valve 106 of the ablation fluid source 102 can be set to the ON position, allowing ablation fluid to flow. The ablation fluid flows from the ablation fluid source 102 to the input port 110 of the heater 104 via the lumen of the flexible tube 108. Upon arriving at the heater 104, the ablation fluid flows between the input port 110 an the output port 112 of the heater 104 via the lumen of the immersed tube. Upon leaving the heater 104 at the output port 112, the ablation fluid is heated to approximately a predetermined temperature (e.g., 60-70 degrees Celsius). The heated ablation fluid flows from the output port 112 of heater 104 to the input port 418 of the ablation applicator 400 via the lumen of the connector tube 116.

In the present example, the temperature controller 120 can control the heater 118 to heat the fluid to a predetermined temperature. The heating element 118 is activated such that the heating liquid in the heater is at or near the predetermined temperature. The sensor 122 can monitor the temperature of ablation fluid in the ablation applicator 400 or the temperature of the application itself or the temperature of the ablation site, and the temperature controller 120 controls the intensity of the heating element 118 such as described herein with respect to FIG. 1.

Once the heated ablation fluid is within the volume of the ablation applicator 400, the heated ablation fluid directly contacts the myocardial ablation site adjacent the right inferior pulmonic vein 502. The contact edge 416 of the ablation applicator 400 thus surrounds an area of the patient's heart muscle defining the ablation site. The suction source 124 will pull the heated ablation fluid through the ablation applicator 400 out the output port 420 of the ablation applicator 400 via the lumen of the output port 420. The heated ablation fluid will continue to flow to the waste reservoir via the lumen of the suction tube 126. When the heated ablation fluid is applied, tissue in contact with the heated ablation fluid (at the ablation site) will increase in temperature. Typically, the cells in such heated tissue are destroyed tissue will die once the tissue is heated to approximately 65 degrees Celsius. When a sufficient amount of tissue has been destroyed, the application of heeled ablation fluid can be turned to the OFF position, such as causing substantially all of the ablation fluid that has been released by the ablation fluid source 102 (both heated and unheated) to flow through the system 100 and into the waste reservoir. When the suction source 124 is deactivated, the ablation applicator 400 can be removed. It is to be understood that the activation and deactivation of the suction source 124 can selectively control flow of heated ablation fluid into and out of the ablation applicator 400.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A system for providing ablation comprising:
a fluid source that contains a volume of biocompatible ablation fluid;
a heater that heats the ablation fluid and provides heated ablation fluid;
an ablation applicator, spaced apart from the heater, and having a perimeter edge configured to engage tissue surround an area of tissue, the ablation applicator applying ablation fluid to the area of tissue; and
a suction source for removing the ablation fluid from the area of tissue.

2. The system of claim 1, further comprising:
temperature sensor that senses a temperature indicative of the heated ablation fluid; and
a temperature control system that adjusts the temperature of the heated ablation fluid based on the sensed temperature.

3. The system of claim 1, wherein the ablation fluid comprises a saline solution.

4. The system of claim 1, wherein the perimeter edge of the ablation applicator defines a circular shape.

5. The system of claim 1, wherein the perimeter edge of the ablation applicator defines a substantially kidney shape.

6. The system of claim 1, wherein the perimeter edge of the ablation applicator defines a substantially C shape.

7. The system of claim 2, wherein the heater further comprises:
a heating element configured to transfer heat to the ablation fluid; and
a tube of a conductive material, spaced apart from the fluid source, that received the ablation fluid from the fluid source; the heating element applying heat to the tube for heating the ablation fluid.

8. A method for performing ablation comprising:
heating, by a heat source, ablation fluid to a predetermined temperature;
providing the heated ablation fluid to an ablation applicator that is spaced apart from the heat source by a conduit;
ablating a tissue area of a patient located within a volume that is defined by the tissue and the ablation applicator; and
removing, by a suction source, heated ablation fluid from the volume as to cause flow of the heated ablation fluid.

9. The method of claim 8, further comprising:
sensing the temperature of the ablation fluid in the ablation applicator; and
adjusting the predetermined temperature of the heated ablation fluid.

10. The method of claim 8, wherein the ablation fluid comprises a saline solution.

11. The method of claim 8, wherein a perimeter edge of the ablation applicator defines a circular shape the method further comprising attaching the perimeter edge of the ablation application to an ablation site on the patient's heart.

12. The method of claim 8, wherein a perimeter edge of the ablation applicator defines a substantially kidney shape, the method further comprising attaching the perimeter edge of the ablation application to an ablation site on the patient's heart.

13. The method of claim 8, wherein a perimeter edge of the ablation applicator defines a substantially C shape, the method further comprising attaching the perimeter edge of the ablation application to an ablation site on the patient's heart.

14. A system for providing ablation comprising:
means for holding/containing a volume of an ablation fluid;
means for heating the ablation fluid;
means for receiving the heated ablation fluid at a location and that is spaced apart from the means for heating or applying the heated ablation fluid to a surface area define by an area of tissue surrounded by a perimeter contact surface of the means for applying;
means for applying negative pressure to remove the heated ablation fluid from the means for applying.

15. The system of claim 14, further comprising means for controlling the temperature of the heated ablation fluid at the surface area.

16. The system of claim 14, wherein the means for applying the heated ablation fluid comprises an ablation applicator having a substantially circular shaped perimeter contact surface.

17. The system of claim 14, wherein the means for applying the heated ablation fluid comprises an ablation applicator having a substantially kidney shaped perimeter contact surface.

18. The system of claim 14, wherein the means for applying the heated ablation fluid comprises an ablation applicator that defines a substantially C shaped perimeter contact surface.
